(19) United States
(12) Patent Application Publication
(10) Pub. No.: US 2006/0147538 A1
(43) Pub. Date: Jul. 6, 2006

(54) Nanocomposite drug delivery composition

(75) Inventors: Duncan Q.M. Craig, Norwich (GB); John Anthony McNally, Lurgan (GB)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: THE QUEEN'S UNIVERSITY OF BELFAST, Belfast (GB)

(21) Appl. No.: 11/267,621

(22) Filed: Nov. 4, 2005

Related U.S. Application Data
(63) Continuation-in-part of application No. PCT/GB04/01931, filed on May 5, 2004.

(30) Foreign Application Priority Data
May 6, 2003 (GB) 0310300.9

(51) Int. Cl. A61K 38/13; A61K 38/28; A61K 31/59; A61K 31/573; A61K 31/405

(52) U.S. Cl. 424/489; 977/906; 514/11; 514/167; 514/3; 514/171; 514/420

(57) ABSTRACT

The invention relates to a drug delivery composition comprising an active ingredient and a biologically inert material wherein the biologically inert material is a nanocomposite material. Preferably the biologically inert material is a polymer-clay nanocomposite comprising up to about 40% by weight of nano-sized (1-1000 nm) clay particles dispersed in a polymeric material. The active ingredient may be dispersed in the nanocomposite material or absorbed thereto.

This is a continuation-in-part of International Application PCT/GB2004/001931 with an international filing date May 5, 2004, the entire disclosure of which is incorporated herein by reference.

The present invention relates to the use of a nanocomposite material in drug delivery compositions.

It is well recognised that there are a number of circumstances whereby it is desirable to disperse a drug in a biologically inert matrix in the preparation of a final dosage form. For example, the incorporation of drugs and bioactive molecules into polymeric matrices (eg implants, solid dispersions) has attracted considerable interest as a means of improved drug delivery. Similarly, drug or bioactive-loaded microspheres and nanospheres have received considerable attention. Various drug delivery compositions comprise modified release systems whereby the drug is released at a controlled rate so as to optimise biological activity and therapeutic effect of the drug (eg controlled release oral drug delivery systems). Another example is the use of drug-loaded medical devices, whereby polymeric devices such as stents may contain antibiotics or anticoagulants for purposes such as the prevention of microbial growth. A further example is the use of tissue engineering scaffolds, whereby growth factors may be incorporated into a polymeric matrix to optimise cell growth on that matrix. In all cases it is necessary to produce systems that not only release the drug at an appropriate rate but also have suitable mechanical properties for the particular application. Nanocomposites are materials that consist of particles of one compound with a mean diameter in the nano-size range (1-1000 nm) dispersed throughout another material, commonly a modified inorganic clay dispersed within an organic polymer. These polymer-clay nanocomposites (PCNs) possess advantageous properties compared to the polymer alone such as increased mechanical strength, reduced gaseous permeability and higher heat resistance, even though the quantity of clay may be 5% or less. Nanocomposite materials have attracted great interest due to the wide range of alterations in the properties of the base polymer engendered by the incorporation of the clays (see for example Schmidt et al, Current Opin. Solid State Mat. Sci. (2002) 6, 205-212; Choi et al, Chem. Mater. (2002) 14, 2936-2939; T. J. Pinnavaia and G. W. Beall, “Polymer-clay nanocomposites”, Wiley, Chichester, 2001). Moreover, they may be manufactured by a range of techniques using equipment that is well established and hence are economical to produce (depending on the choice of materials, although commonly the materials used are well recognised and inexpensive).

The use, in drug delivery compositions, of potentially useful matrix materials can be limited by their mechanical properties. The matrix must maintain suitable mechanical integrity during the course of the manufacture process and through its subsequent handling and use.

There are many instances whereby the mechanical properties and/or the release rate of the drugs or bioactives of known drug delivery compositions are sub-optimal. The present invention providing as it does for drug or bioactive-loaded nanocomposites seeks to address these difficulties.

Therefore, it is an object of the present invention to provide a drug delivery composition wherein the release rate of the drug may be manipulated or altered so as to be optimised for a given drug or application.

It is another object of the invention to provide a drug delivery composition which is mechanically suitable for the application to which the drug delivery composition is to be put and which is capable of maintaining mechanical integrity throughout the course of its manufacture, storage, handling and use as appropriate.

It is a further object of the invention to provide a drug delivery composition the manufacture of which may be carried out economically viable using equipment that is readily available.

Accordingly, the present invention provides for the use of a nanocomposite material in the manufacture of a drug delivery composition.

The invention also provides a drug delivery composition comprising an active ingredient and a biologically inert material wherein the biologically inert material is a nanocomposite material, preferably a polymer-clay nanocomposite.

Preferably the active ingredient is dispersed throughout a matrix comprising the biologically inert material, although the invention also provides a drug delivery system wherein the active ingredient is loaded in, or adsorbed to, a vehicle comprising the biologically inert material.

The invention further provides a method of manufacturing a drug delivery composition comprising the steps of forming an admixture comprising a polymer, a clay and an active ingredient and extruding the admixture to produce an extrudate.

The nanocomposite material may comprise up to about 99.9% w/w polymer. Preferably the polymer is present in an amount of from about 90% w/w to about 99% w/w of the nanocomposite.

A wide range of polymers may be employed in the biologically inert material. Examples of suitable polymers include polyethylene glycol, poly(ε-caprolactone), polyvinylpyrrolidone, polylactide, polyethylene, polystyrene, poly(dimethylsiloxane), polyaniline, polyester, polyimide, cellulose derivatives such as hydroxyproyl methyl cellulose and ethylcellulose, polysaccharides such as alginates and chitosans, gelatin, polymethylmethacrylates, silicones, polyacrylonitrile, polyetheretherketone (PEEK), polyamide, polyurethane, bone and dental cements and other polymeric prosthetic materials. In addition materials such as starch and starch derivatives would also be suitable for use in the inert material. Materials that are composed of more than one polymer or a polymer and a plasticizer such as polyethylene glycol, water or glycerol may also be included.

Typically the level of clay within the nanocomposite may range from less than 1% w/w to about 40% w/w, although higher levels may be included. Preferably the amount of clay in the nanocomposite is within the range of from 1% w/w to 10% w/w of the nanocomposite material.

Various clays may be used, either alone or in combination. Typically silicates may be used that may be naturally occurring (for example bentonite, montmorillonite and other smectites) or synthetic (for example fluorohectorite, fluoromica, layered double hydroxides).

The presence of the clay nanoparticles can dramatically alter the mechanical properties of the composition of the invention, compared to a conventional drug delivery vehicle using a polymer-only matrix, so as to render the system much more suitable for a particular application. The mechanical properties of the drug delivery composition of the invention may be manipulated by suitable choice of nanocomposite component materials (ie the polymers and clays used) and/or manufacturing conditions. Furthermore, the rate at which the composition biodegrades may differ from that of the polymer alone and may be tailored to suit a particular active ingredient or therapeutic application.

The teaching of the invention is applicable to all such methods of nanocomposite manufacture and to all active ingredients (drugs and bioactive materials including growth factors, nutraceuticals, antimicrobials and the like) which can withstand the manufacturing conditions. Suitable drugs and bioactives include for example low molecular weight compounds such as indomethacin and paracetamol, higher molecular weight compounds such as hydrocortisone, peptides such as cyclosporin A and calcitonin and proteins such as insulin and human recombinant DNAse. The manufacturing method used may be tailored to suit both the performance requirements of the composition and the lability of the incorporated bioactive such that degradation may be minimised by appropriate choice of manufacturing method.

The amount of active ingredient employed in the drug delivery composition of the present invention may vary depending on the characteristics of each particular agent. However, the active ingredient should be employed in an amount which is sufficient to elicit a therapeutic response upon release from the drug delivery composition. Typically the active ingredient may be employed in an amount of from less than 1% to about 40% by weight of the composition.

A drug delivery composition of the invention may be prepared according to any known method of manufacturing nanocomposites which can be modified so as to facilitate the incorporation of the drugs or bioactive molecule, for example by melt extrusion. Other manufacturing methods include in situ polymerisation (Paul et al, (2003) Polymer, 44, 443-450), melt intercalation (Lepoittevin et al (2002) Polymer 43, 4017-4023), sonication (Burnside and Giannelis (1995) Chemistry of Materials, 7, 1597-1600) sol-gel technology and solution blending.

In the case of manufacture by melt extrusion, the various components may be mixed simultaneously (prior to extrusion) in order to disperse the active ingredient throughout the nanocomposite material, although the mixing sequence can influence the product structure and performance and represents another means by which the properties and release characteristics of the composition may be controlled. However, premixing is not essential. Indeed, it may be more effective to add the clay and drug after the polymer has been added and melted in the extruder. An additive may be added together with the mixture or separately further down the length of the extruder. Generally, addition of additives down the length of the extruder, i.e. separate from the polymer gives better mixing.

As the skilled person will be aware many different types of extruder exist. The number of heating zones present depends on the type of extruder.

In one embodiment of the invention the screw configuration used has 5 heating zones, a reverse element and up to 5 kneading blocks.

In an embodiment of the invention a co-rotating intermeshing twin screw extruder is used.

The polymer may be added in pellitized form. However, it is not essential that the polymer is pellitized.

Other factors such as the choice of extrusion screw geometries may influence the structure and performance of the extrudate.

Typically a screw configuration is utilised which optimises mixing of the components. For example, in an embodiment of the invention the screw has a reverse element which reverses the flow back over 3 kneading blocks with 2 further kneading blocks in the final sections. For the temperatures utilised in melt processing in the present invention a slow screw speed of around 10 rpm to 150 rpm may be used.

As the nanocomposite exists in the extruder, it can be made into a sheet, film, tube or pelletised or extruded into other shapes.

The drug-loaded nanocomposite extrudate produced may be ground and then formulated into dosage forms such as tablets and capsules. In such cases, the person skilled in the art would appreciate that excipients such as diluents, lubricants, glidants, disintegrants and the like may be utilised in preparation of the final dosage form. Further modifications known in the field of formulation chemistry, such as the application of enteric or taste masking coatings to tablets for example, may be employed.

Dosage forms categories for which the invention may be particularly useful include oral drug delivery systems for modified (fast or slow) release, implant systems (biodegradable or non-biodegradable), microspheres and nanoparticles for oral, nasal, parenteral or topical delivery, medical devices, suppositories, pessaries, dermatological preparations, tissue engineering scaffolds.

The present invention also provides a drug delivery system wherein an active ingredient loaded in, or adsorbed to, a vehicle comprising the biologically inert material, the biologically inert material being a nanocomposite material. The use of nanocomposites in the manufacture of drug-loaded medical devices (for example devices such as stents containing antibiotics or anticoagulants) affords similar advantages as those discussed above in terms of controlled active ingredient delivery and robustness.

Embodiments of the present invention will now be described by way of example only with reference to the accompanying drawings, in which:

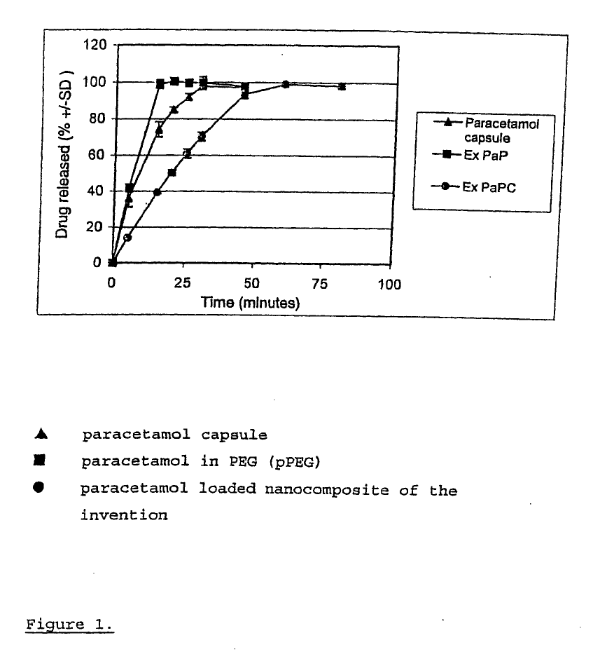

FIG. 1 illustrates the release profile of the three combinations tested, as discussed in Example 1;

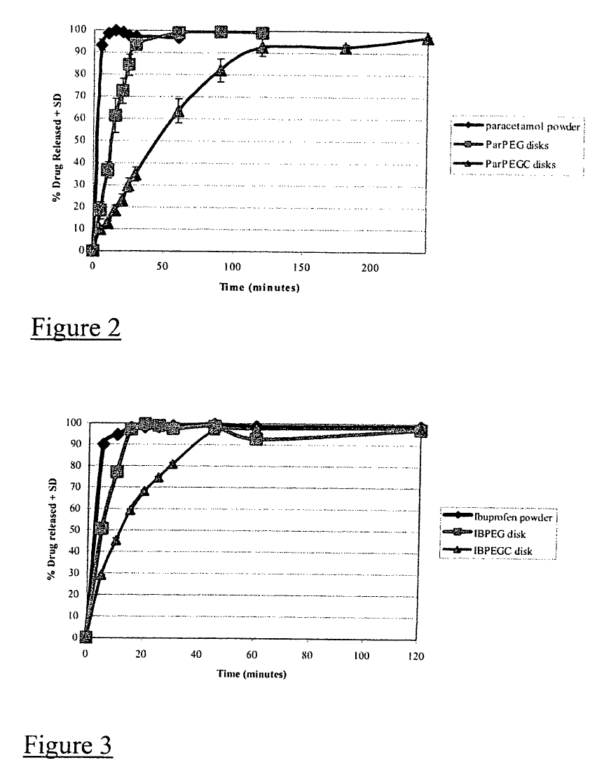

FIG. 2 illustrates the release profile of paracetamol powder, paracetamol and PEG discs, and paracetamol, PEG and clay nanocomposite discs;

FIG. 3 illustrates the release profile of ibuprofen powder, ibuprofen and PEG discs, and ibuprofen, PEG and clay nanocomposite discs;

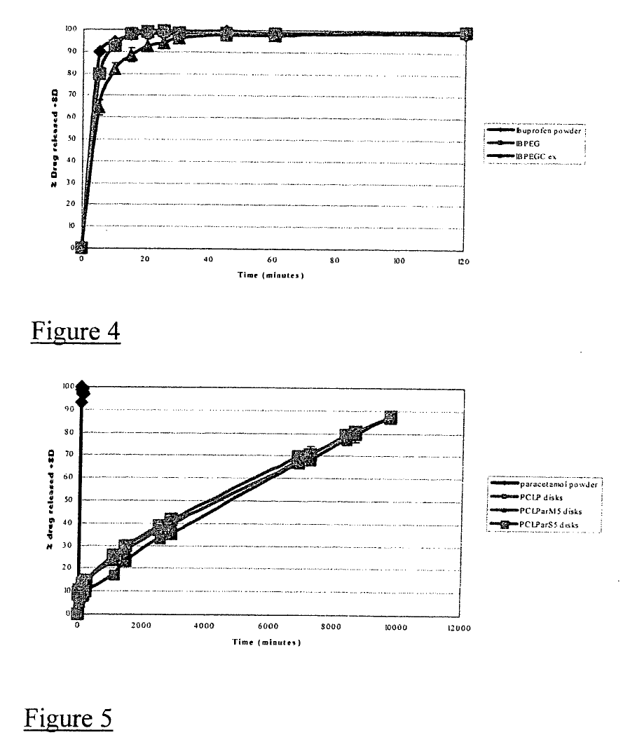

FIG. 4 illustrates the release profile of ibuprofen powder, ibuprofen and PEG, and ibuprofen, PEG and clay nanocomposite extrudates;

FIG. 5 illustrates the release profile of paracetamol powder, paracetamol and polycaprolactone discs, paracetamol and polycaprolactone and montmorillonite (natural clay) at 5% nanocomposite discs, and paracetamol and polycaprolactone and synthetic clay at 5% nanocomposite discs;

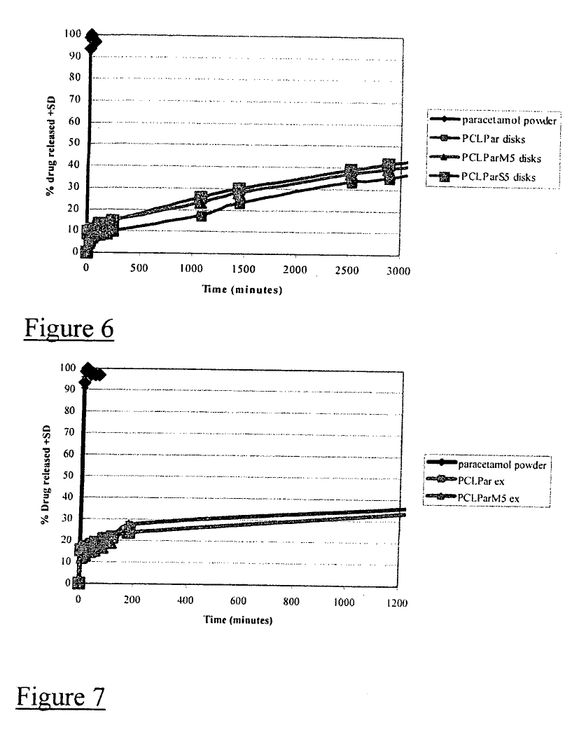

FIG. 6 illustrates the release profile of paracetamol powder, paracetamol and polycaprolactone discs, paracetamol and polycaprolactone and montmorillonite (natural clay) at 5% nanocomposite discs, and paracetamol and polycaprolactone and synthetic clay at 5% nanocomposite discs over the initial stages of release;

FIG. 7 illustrates the release profile of paracetamol powder, paracetamol and polycaprolactone extrudate, and paracetamol and polycaprolactone and montmorillonite (natural clay) at 5% extrudate; and

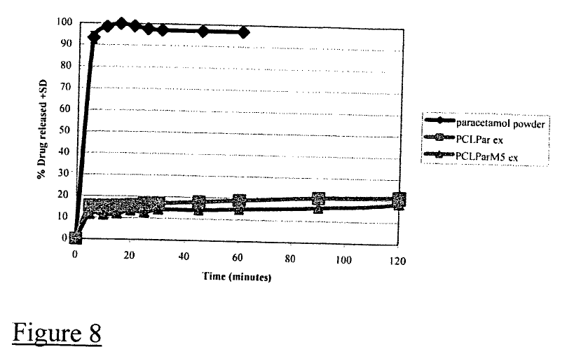

FIG. 8 illustrates the release profile of paracetamol powder, paracetamol and polycaprolactone extrudate, and paracetamol and polycaprolactone and montmorillonite (natural clay) at 5% extrudate over the initial stages of release.

### EXAMPLE 1

Drug dispersions in polyethylene glycol based nanocomposites for the oral administration of drugs were prepared as follows:

Polyethylene glycol (PEG) 20000 (Janssen Pharmaceuticals) was the polymer employed and Cloisite 30B (Southern Clay Products, USA) was the clay component. Paracetamol (Sigma, UK) was used as a model active ingredient. Production of the nanocomposites was performed by melt extrusion using a Killon KN-100 (Davis Standard Corporation, USA) single screw extruder with rod shaped die (38 mm screw diameter, speed 20-22 rpm, die temp 54-57° C., temperature zone 1 50° C.—temperature zone 2 55-60° C.—temperature zone 3 55-60° C.—temperature zone 4 55-60° C., haul off speed 3-4 m/min, cool to room temperature). The powders were not subjected to any treatments prior to extrusion, other than simple mixing of the three components simultaneously.

The following combinations were used (all % values are percentages by weight:
Paracetamol capsule (number 3, white, gelatin capsule)
5% paracetamol in PEG (pPEG)
paracetamol 5%/Cloisite 30B 4%/PEG 95% (the drug loaded nanocomposite of the invention)

The extrudates emerged as cylindrical solid tube-like structures of approximately 5 mm in diameter. During the processing of pPEG the following readings were obtained: screw amps: 4; die pressure: 0.1 kg/cm²; however when the nanocomposite mixture was extruded the screw amps and die pressure values increased to 8 and 0.4 respectively evidencing the enhanced mechanical strength and resistance of the nanocomposites. Extrusion conditions were optimised by initially heating the system to beyond the melting point of the PEG (circa 60° C.) and cooling to circa 56° C. so as to extrude the material when in a supercooled state thus facilitating rapid solidification upon extrusion from the equipment. The nanocomposite extrudates produced were mechanically robust and could be snapped by manual application of pressure.

In testing the release characteristics of each sample the following dissolution methodology was used (Copley DIS 8000): USP apparatus 2—rotating paddle, 50 rpm; medium—900 ml deionised water (37° C.±0.5° C.); analysis—UV spectrophotometer (243 nm).

Dissolution properties were measured as follows: A UV calibration plot from a stock solution of paracetamol was prepared (100 mg in 100 ml), with measurements taken at 249 nm. Five samples were used for each experiment with 10 ml removed at appropriate time intervals and replaced with 10 mls 37° C. deionised water. The samples were analysed using UV measurement at 249 nm. Samples were prepared by breaking the extrudate into approximately 1 cm lengths, with a corresponding sample weight of circa 0.3 g. For the pPEG samples, samples were taken every 5 minutes for 30 minutes. For the nanocomposite composition samples were taken every 20 minutes for 4 hours.

The release profiles of the three combinations tested are shown in FIG. 1. The release profile of the paracetamol nanocomposite of the invention indicates a slower release rate plateauing at about 60 min compared to rate of release from the paracetamol capsule which reached a plateau at about 30 min. The release profile of the pPEG sample was faster that both the drug loaded nanocomposite of the invention and the paracetamol capsule, plateauing after about 20 min.

The test data indicates that the nanocomposite system may be used as a controlled release drug delivery system whereby drug release from the composition is slowed or otherwise manipulated in comparison to the non-clay containing system.

### EXAMPLE 2

A further drug delivery composition, in the form of a drug loaded polyurethane nanocomposite for use in an insert device, was prepared as follows:

The polymer/clay/drug composition was thermoplastic polyurethane (95%)/Cloisite 30B (4%)/hydrocortisone (1%). The mixture of constituents was extruded using a Collin GmbH twin screw extruder (Model ZK 25), adapter temperature 190° C., die temperature 19° C., melt temperature 188° C., melt zones on the extruder were set between 195° C. and 190° C. from the feed end and screw speed was 90 rpm. The mixture was extruded through a cast film die to produce 200 micron thick, 40 to 50 mm wide film of the drug loaded nanocomposite.

### EXAMPLE 3

Further drug delivery compositions in the form of PEG or polycaprolactone nanocomposites were prepared as follows:

1. Loading of Drug and Clay

A first mixture comprising Polycaprolactone (PCL) with 5 wt % model drug (paracetamol, ibuprofen) with a clay loading of 0, 1, 3 or 5 wt % clay (natural or synthetic) was prepared.

A second mixture comprising Polyethylene glycol (PEG) (molecular weight=20,000) with 5 wt % model drug (paracetamol, ibuprofen) with a clay loading of 0, 1, 3, or 5 wt % clay (natural or synthetic) was prepared.

In each case the drug, polymer and/or clay was premixed before processing.

2. Processing Conditions

The same screw configuration and die was used for the PCL and PEG mixtures. However, the heating zones of the system were different and these are listed below.

In this example the PCL used was pelletized, and the pellet of the PCL was round in shape. The PEG was not pelletized.

The screw configuration was designed to have 3 kneading zones.

Polycaprolactone

The system used to provide the nanocomposite which includes polycaprolactone was a Collin Zk25 twin screw extruder, with 6 heater zones set at 70° C., 80° C., 80° C., 75° C., 70° C. and 60° C. It will be appreciated by those skilled in the art that slight variations in these temperatures may be used, for example +/−2° C. The screw speed is 60 rpm, Die 3 mm. The extrudate is air cooled by a gun blowing air onto the extrudate just after the die entrance and further cooled along a conveyor belt (Collin CR 136/350) and then pelletized using a Collin teach-line CSG 1715). The loading of the paracetamol was 5 wt % and the loading of the clays was 0, 1, 3, or 5 wt %.

Polyethylene Glycol

The system used to provide the nanocomposite which includes polyethylene glycol (molecular weight=20,000) Collin Zk25 twin screw extruder with 6 heater zones set at 55° C., 60° C., 60° C., 60° C., 60° C., 60° C. and 60° C. The screw speed is 60 rpm, Die 3 mm. As above, it will be appreciated by those skilled in the art that slight variations in temperature may be used. The extrudate was air-cooled along a conveyor belt. The loading of the paracetamol was 5 wt % and the loading of the clays was 0, 1, 3, or 5 wt %.

As will be appreciated by those skilled in the art the drugs paracetamol and ibuprofen described in the present example are provided as model drugs. These drugs have been described due to their availability and cost, however, other drugs may be used with the described nanocomposites. It will be appreciated that different release rates may be observed for different drugs due to the different solubility of said drugs.

3. Release Studies

Discussion Drug Release PEG-Paracetamol-Clay Disks

| | Time 50% | Time 80% | Time 100% |
|---|---|---|---|
| | release | release | release |
| Sample | (minutes) | (minutes) | (minutes) |
| Parcetamol | <5 minutes | <5 minutes | <5 minutes |
| powder | | | |
| PEGPar | 12.5 | 23 | 45 |
| PEGParM1 | 13 | 23 | 45 |
| PEGParM3 | 20 | 35 | 120 |
| PEGParM5 | 40 | 180 | 300 |
| PEGParS1 | 30 | 45 | 120 |
| PEGParS3 | 40 | 90 | 300 |
| PEGParS5 | 45 | 95 | 300 |

Discussion Drug Release PEG-Ibuprofen-Clay-Disks

| | Time 50% | Time 80% | Time 100% |
|---|---|---|---|
| | release | release | release |
| Sample | (minutes) | (minutes) | (minutes) |
| Ibuprofen | <5 | <5 | 10 |
| Powder | | | |
| PEGIB | 5 | 10.5 | 15 |
| PEGIBM1 | 5 | 10.5 | 15 |
| PEGIM3 | 5 | 10.5 | 15 |
| PEGIBM5 | 5.5 | 11 | 15 |
| PEGIBS1 | 5 | 12 | 20 |
| PEGIBS3 | 12.5 | 25 | 60 |
| PEGIBS5 | 12.5 | 30 | 60 |

PEGIB is PEG+ibuprofen, PEGIBM is PEG+ibuprofen+montmorillonite (natural clay) and PEGIBS is PEG+ibuprofen+synthetic clay.

In a one embodiment the discs were 18 mm in diameter and 1 mm thick. Different sizes or shapes of discs will influence the release rate of the drug.

Discussion Drug Release PEG-Paracetamol-Clay Extrudate

| | Time 50% | Time 80% | Time 100% |
|---|---|---|---|
| | release | release | release |
| Sample | (minutes) | (minutes) | (minutes) |
| Paracetamol | <5 minutes | <5 minutes | <5 minutes |
| powder | | | |
| PEGPar | 2 | 3.25 | 6 |
| PEGParM1 | 2 | 3.25 | 6 |
| PEGParM3 | 2 | 3.25 | 6 |
| PEGParM5 | 2 | 3.25 | 6 |
| PEGParS1 | 2.5 | 4.5 | 60 |
| PEGParS3 | 6 | 17 | 60 |
| PEGParS5 | 5 | 19 | 120 |

Discussion Drug Release PEG-Ibuprofen-Clay-Extrudate

| | Time 50% | Time 80% | Time 100% |
|---|---|---|---|
| | release | release | release |
| Sample | (minutes) | (minutes) | (minutes) |
| Ibuprofen | <5 | <5 | 10 |
| Powder | | | |
| PEGIB | 2.5 | 5 | 5 |
| PEGIBM1 | 3 | 6 | 15 |
| PEGIBM3 | 3 | 6 | 15 |
| PEGIBM5 | 3 | 6 | 25 |
| PEGIBS1 | 2.5 | 5 | 15 |
| PEGIBS3 | 3.5 | 11 | 30 |
| PEGIBS5 | 3.75 | 9 | 25 |

PEGIB is PEG+ibuprofen, PEGIBM is PEG+ibuprofen+montmorillonite (natural clay) and PEGIBS is PEG+ibuprofen+synthetic clay. In one embodiment the extrudate is rod shaped with a diameter of 1 mm.

Discussion Drug Release PCL-Paracetamol-Clay Extrudate

| | Time 50% | Time 80% | Time 100% |
|---|---|---|---|
| | release | release | release |
| Sample | (minutes) | (minutes) | (minutes) |
| Paracetamol | <5 minutes | <5 minutes | <5 minutes |
| powder | | | |
| PCLPar | >1260 | >1260 | >1260 |
| PCLParM5 | >1260 | >1260 | >1260 |

Discussion Drug Release PCL-Paracetamol-Clay Disks

| | Time 50% | Time 80% | Time 100% |
|---|---|---|---|
| | release | release | release |
| Sample | (minutes) | (minutes) | (minutes) |
| Paracetamol | <5 minutes | <5 minutes | <5 minutes |
| powder | | | |
| PCLPar | 3800 | 8400 | >9720 |
| PCLParM5 | 4200 | 8400 | >9720 |
| PCLParS5 | 4600 | 8400 | >9720 |

PCLPar is polycaprolactone+paracetamol, PCLParM is polycaprolactone+paracetamol+montmorillonite (natural clay) and PCLParS is polycaprolactone+paracetamol+synthetic clay.

In one embodiment the extrudate is rod shaped with a diameter of 1 mm.

The above results indicate that drug release rate is significantly retarded on addition of nanoclay to the polymer/drug mix. The retardation effect is greater with increasing nanoclay loading and synthetic nanoclay is better than the natural clay at retarding the drug release rate.

Various improvements and modifications may be made without departing from the scope of the present invention.

1. A method of manufacturing a drug delivery composition comprising an active ingredient and a biologically inert material which is a nanocomposite material comprising the steps of
forming an admixture comprising a polymer, a clay and active ingredient, and
extruding the admixture to produce an extrudate.

2. The method of manufacturing a drug delivery composition as claimed in claim 1 wherein the step of extruding is melt extrusion.

3. A method of manufacturing a drug delivery composition as claimed in claim 1 wherein the nanocomposite comprises at least one polymer selected from the group consisting of polyethylene glycol, poly(ε-caprolactone), polyvinylpyrrolidone, polylactide, polyethylene, polystyrene, poly(dimethylsiloxane), polyaniline, polyester, polyimide, cellulose derivatives such as hydroxyproyl methyl cellulose and ethylcellulose, polysaccharides such as alginates and chitosans, gelatin, polymethylmethacrylates, silicones, polyacrylonitrile, PEEK, polyamide, polyurethane, bone and dental cements, starch and starch derivatives.

4. A method of manufacturing a drug delivery composition as claimed in claim 1 wherein the nanocomposite comprises at least one clay selected from the group consisting of bentonite, montmorillonite, fluorohectorite, fluoromica and layered double hydroxides.

5. A method of manufacturing a drug delivery composition as claimed in claim 1 wherein the amount of clay within the nanocomposite is up to 40% w/w of the nanocomposite material.

6. A method of manufacturing a drug delivery composition as claimed in claim 1 wherein the at least one active ingredient is selected from the group consisting of indomethacin, paracetamol, hydrocortisone, cyclosporin A, calcitonin, insulin and human recombinant DNAse.

7. A method of manufacturing a drug delivery composition as claimed in claim 1 wherein the active ingredient is present in an amount of up to 40% by weight of the drug delivery composition.

8. A drug delivery composition comprising an active ingredient and a biologically inert material which is a nanocomposite material obtained from a method comprising the steps:
forming an admixture comprising a polymer, a clay and active ingredient, and
extruding the admixture to produce an extrudate.

9. A drug delivery composition as claimed in claim 8 wherein the nanocomposite comprises at least one polymer selected from the group consisting of polyethylene glycol, poly(ε-caprolactone), polyvinylpyrrolidone, polylactide, polyethylene, polystyrene, poly(dimethylsiloxane), polyaniline, polyester, polyimide, cellulose derivatives such as hydroxyproyl methyl cellulose and ethylcellulose, polysaccharides such as alginates and chitosans, gelatin, polymethylmethacrylates, silicones, polyacrylonitrile, PEEK, polyamide, polyurethane, bone and dental cements, starch and starch derivatives.

10. A drug delivery composition as claimed in claim 8 wherein the nanocomposite comprises at least one clay selected from the group consisting of bentonite, montmorillonite, fluorohectorite, fluoromica and layered double hydroxides.

11. A drug delivery composition as claimed in claim 8 wherein the amount of clay within the nanocomposite is up to 40% w/w of the nanocomposite material.

12. A drug delivery composition as claimed in claim 8 wherein the at least one active ingredient is selected from the group consisting of indomethacin, paracetamol, hydrocortisone, cyclosporin A, calcitonin, insulin and human recombinant DNAse.

13. A drug delivery composition as claimed in claim 8 wherein the active ingredient is present in an amount of up to 40% by weight of the drug delivery composition.
